SEQUENCE LISTING (1 sequence)

SEQ ID NO: 1 - protein, 1296 aa, Clostridial botulinum A
  DOMAIN (1)...(448): Light chain, enzymatic domain; therapeutic domain
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

(19) United States
(12) Patent Application Publication
(10) Pub. No.: US 2013/0330369 A1
(43) Pub. Date: Dec. 12, 2013

(54) Reduction Of Antibody Response Against Botulinum Neurotoxin And Variants Thereof

(71) Applicants: M. Zouhair Atassi, Houston, TX (US); Behzod Z. Dolimbek, Houston, TX (US); K. Roger Aoki, Coto de Caza, CA (US)

(72) Inventors: M. Zouhair Atassi, Houston, TX (US); Behzod Z. Dolimbek, Houston, TX (US); K. Roger Aoki, Coto de Caza, CA (US)

(73) Assignee: Allergan, Inc., Irvine, CA (US)

(21) Appl. No.: 13/878,288

(22) PCT Filed: Oct. 7, 2011
(86) PCT No.: PCT/US2011/055407

§ 371 Date: Jun. 7, 2013

Related U.S. Application Data
(60) Provisional application No. 61/391,231, filed on Oct. 8, 2010.

(51) Int. Cl. A61K 47/48; A61K 39/08

(52) U.S. Cl. CPC A61K 47/48215; A61K 39/08
USPC 424/190.1

(57) ABSTRACT

The present invention provides a method of tolerizing a subject to botulinum toxin and botulinum toxin variants.

This is a national stage application under 35 U.S.C. §371 of PCT patent application PCT/US11/055407, filed on Oct. 7, 2011, which claims the benefit of U.S. provisional patent application Ser. No. 61/391,231, filed Oct. 8, 2010, entirely incorporated by reference.

### FIELD

The present invention relates to methods for modulating antibody responses against botulinum neurotoxins and variants thereof.

### BACKGROUND

Botulinum neurotoxins (BoNTs) such as BoNT/A, BoNT/B, etc., act on the nervous system by blocking the release of acetylcholine (ACh) at the pre-synaptic neuromuscular junction. The action of BoNT is initiated by its binding to a receptor molecule on the cell surface, then the toxin-receptor complex undergoes endocytosis. Once inside the cell, the toxin blocks ACh release. The binding of BoNT's A and B to the cell membrane, which is a function of the H (heavy) chain, enables the L (light) chain (which is a zinc endopeptidase) or a combination of H and L chains, to be internalized and cause paralysis (Aoki et al., 2010).

Because of their action at the presynaptic neuromuscular junction, BoNTs types A and B have been employed to treat a variety of neuromuscular disorders, including cervical dystonia (CD), and in cosmetic and other therapeutic applications (Atassi and Oshima, 1999; Jankovic, 2002, 2004). There is no cure for CD, but injection with BoNT (usually type A or B) into the affected muscle(s) at 3 to 6-month intervals is widely used to treat the disorder (Naumann et al., 1998). However, repeated injections can elicit blocking (neutralizing) antibodies (Abs) against BoNT/A, which can reduce or completely eliminate the patient's responsiveness to further treatment (Naumann et al., 1998; Atassi, 2004; Göschel et al., 1997; Jankovic, 2002; Greene et al., 1994). Should that happen, the toxin used for clinical treatment is often changed from type A to type B (BoNT/B) (Dressler et al., 2003; Comella et al., 2005). However, to maintain clinical effectiveness when using type B, its administration is required more frequently and at higher doses than type A (Sloop et al., 1997), which stimulates the appearance of Abs against BoNT/B and frequently leads to Ab-induced therapy failure (Dresslerand and Bigalke, 2004). Therefore, to prolong the period of effective treatment it would be beneficial to suppress the effect of blocking Abs.

Variants of BoNT, such as BoNT/A retargeted to sensory nerves by replacing part of the H chain with a sensory nerve binding moiety, are currently in clinical trials for treatment of pain and other disorders. See, for example, U.S. Pat. Nos. 5,989,545; 6,962,703; 6,461,617; 7,244,437; and 7,244,436, as well as U.S. patent application Ser. Nos. 11/829,118 and 12/308,078, all entirely incorporated by reference. Similar to BoNT/B, these retargeted molecules will likely be dosed at higher levels and thus may lead to Ab responses at a higher rate than BoNT/A.

Tolerization of a mammal to immunogenic proteins has been taught previously by Dr. Atassi, one of the present inventors. See, e.g., U.S. Pat. Nos. 6,048,529; 7,531,179; 7,341,843; 7,635,484; and, 7,968,304, all entirely incorporated by reference.

### BRIEF DESCRIPTION OF THE FIGURES

FIG. 1. Percent survival in MPA as a function of titers of protective Abs in anti-BoNT/A antisera (test bleed 1) of mice tolerized with mPEG-conjugates of peptide N8, N25, C15, or C31 of BoNT/A. Ab responses in mPEG-peptide (10 μg per dose) tolerized (groups 1-4) and non-tolerized mice (group 5) were assayed by MPA in 5 mice at each antiserum dilution shown. The number of mice (in percent) that survived challenge with 1.05×LD₁₀₀ of active BoNT/A are plotted. The decrease in Ab response measured by RIA is given in parentheses.

FIG. 2. Percent survival in MPA as a function of titers of protective antibodies in anti-BoNT/A antisera (test bleed 2) from mice tolerized with mPEG-conjugates of peptide N8, N25, C15 or C31. Ab responses of mPEG-peptide (10 μg per dose) tolerized (groups 1-4), and non-tolerized (group 5) mice against BoNT/A were assayed by MPA in 5 mice at each antiserum dilution shown. The number of mice (in percent) that survived challenge with 1.05×LD₁₀₀ of active BoNT/A are plotted.

FIG. 3. Survival of mice in MPA as a function of titers of protective Abs in anti-BoNT/A antisera (test bleed 1) from mice tolerized with 30 μg per dose of mPEG-conjugates of peptide N8, N25, C15 or C31 (groups 1-4) of BoNT/A., and non-tolerized (group 5) mice. After tolerization with mPEG-peptide mice were immunized with 1 μg of BoNT/A and the antisera were assayed by MPA in 5 mice at each antiserum dilution. The number of mice in each group (in percent) that survived challenge with 1.05×LD₁₀₀ of active BoNT/A are plotted. The decrease in Ab response determined by RIA are shown in curly brackets.

FIG. 4. Survival of mice in MPA as a function of titers of protective Abs in anti-BoNT/A antisera (test bleed 2). Mice were tolerized with 30 μg mPEG conjugates of peptide N8, N25, C15 or C31 of BoNT/A (groups 1-4), and non-tolerized (group 5). The mice were immunized with 1 μg of BoNT/A and the antisera were assayed by MPA in 5 mice at each antiserum dilution. The number of mice in each group (in percent) that survived challenge with 1.05×LD₁₀₀ of active BoNT/A are plotted.

FIG. 5. Survival of mice in MPA as a function of titers of protective Abs in anti-BoNT/A antisera obtained at day 142 post-immunization (test bleed 3). Mice were tolerized by administering three i.p. injections of 30 μg each of mPEG conjugates of peptides N8, N25, C15 or C31 (groups 1-4) at days −11, −7 and −3. Tolerized and non-tolerized controls (group 5) were immunized with 1 μg of BoNT/A and two boosters of a similar dose on days 21 42. The mice were re-tolerized with 30 μg each of the correlate mPEG-peptide on days 121, 125 and 129. The mice received a booster injection (1 μg) of BoNT/A on day 132. The antisera were obtained on day 142 and assayed by MPA in 5 mice at each antiserum dilution. The number of mice in each group (in percent) that survived challenge with 1.05×LD₁₀₀ of active BoNT/A are plotted. Values on each curve represent the antiserum dilution at 50% survival and the value in parentheses represents the decrease in antibody titer relative to controls (group 5).

FIG. 6. Protective activity of the antisera after short-term (test bleeds 1 and 2 on days 31 and 52, respectively) and long-term (test bleed 3 on day 142). In test bleed 2 the blocking activity of the Abs had increased and was less than 10% lower than controls. Then after a second treatment with mPEG-peptide (3×30 μg) and re-immunization with toxin a third test bleed was obtained on day 142. Protective Ab levels in antisera decreased and 50% percent survival was obtained at a lower dilution of the antisera. Values on each bar represent the dilution at which 50% survival was obtained.

### DESCRIPTION

It has been discovered that mPEG coupled to the N-terminal amino group of specific BoNT sequences are useful to tolerize a mammal to BoNT.

Abbreviations used in this document: Ab means antibody; BoNT/A means botulinum neurotoxin serotype A; BoNT-derived peptide means a peptide whose amino acid sequence is similar to a discreet portion of the amino acid sequence of BoNT; BSA means bovine serum albumin; DMF means dimethylformamide; H-chain means the heavy chain (residues 449-1296) of BoNT/A; ICR means imprinted control region; Inoculate means to introduce a substance within the body of a subject via any suitable mechanism, such as, for example, injection, or the like; i.p. means intraperitoneally; mPEG means monomethoxypolyethylene glycol; MPA means mouse protection assay; PBS means 0.15M NaCl in 0.01M sodium phosphate buffer, pH 7.20; peptide numbers preceded by C indicate C-terminal domain (H_{C}; residues 855-1296) peptides of BoNT/A; peptides denoted by N indicate N-terminal domain (H_{N}; residues 449-859) peptides of the H-chain of BoNT/A; s.c. means subcutaneously; and snp means synaptosome.

As used herein, the term “naturally occurring BoNT/A toxin variant” means any BoNT/A toxin produced without the aid of any human manipulation, including, without limitation, BoNT/A toxin isoforms produced from alternatively-spliced transcripts and BoNT/A toxin isoforms produced by spontaneous mutation. As used herein, the term “non-naturally occurring BoNT/A toxin variant” means any BoNT/A toxin produced with the aid of human manipulation, including, without limitation, a BoNT/A toxin produced by genetic engineering using random mutagenesis or rational designed and a BoNT/A toxin produced by chemical synthesis.

As used herein, the term “BoNT/A toxin variant,” whether naturally-occurring or non-naturally-occurring, means a BoNT/A toxin that has at least one amino acid change from the corresponding region of SEQ ID NO: 1 and can be described in percent identity to the corresponding region of SEQ ID NO: 1. As a non-limiting example, a BoNT/A toxin variant comprising amino acids 1-1296 of SEQ ID NO: 1 will have at least one amino acid difference, such as, e.g., an amino acid substitution, deletion or addition, as compared to the amino acid region 1-1296 of SEQ ID NO: 1. As another non-limiting example, a BoNT/A toxin variant comprising amino acids 449-1296 of SEQ ID NO: 1 will have at least one amino acid difference, such as, e.g., an amino acid substitution, deletion or addition, as compared to the amino acid region 449-1296 of SEQ ID NO: 1. As yet another non-limiting example, a BoNT/A toxin variant comprising amino acids 861-1296 of SEQ ID NO: 1 will have at least one amino acid difference, such as, e.g., an amino acid substitution, deletion or addition, as compared to the amino acid region 861-1296 of SEQ ID NO: 1. Any of a variety of sequence alignment methods can be used to determine percent identity, including, without limitation, global methods, local methods and hybrid methods, such as, e.g., segment approach methods.

As used herein, the term “BoNT/A toxin chimeric variant” means a molecule comprising at least a portion of a BoNT/A toxin and at least a portion of at least one other protein to form a BoNT/A toxin. Such BoNT/A toxin chimeric molecules are described in, e.g., Clifford C. Shone et al., Recombinant Toxin Fragments, U.S. Pat. No. 6,461,617 (Oct. 8, 2002); Keith A. Foster et al., Clostridial Toxin Derivatives Able To Modify Peripheral Sensory Afferent Functions, U.S. Pat. No. 6,395,513 (May 28, 2002); Wei-Jin Lin et al., Neurotoxins with Enhanced Target Specificity, US 2002/0137886 (Sep. 26, 2002); Keith A. Foster et al., Inhibition of Secretion from Non-neural Cells, US 2003/0180289 (Sep. 25, 2003); J. Oliver Dolly et al., Activatable Recombinant Neurotoxins, WO 2001/014570 (Mar. 1, 2001); Clifford C. Shone et al., Recombinant Toxin Fragments, WO 2004/024909 (Mar. 25, 2004); Keith A. Foster et al., Re-targeted Toxin Conjugates, WO 2005/023309 (Mar. 17, 2005), and US2009/0069238 (Aug. 15, 2008); J. Oliver Dolly et al., Activatable Clostridial toxins; all entirely incorporated by reference.

Animals, Botulinum Neurotoxin and Synthetic Peptides

Outbred (ICR) mice 7-9 weeks old, weighing 22-24 g were obtained from The Center for Comparative Medicine, Baylor College of Medicine. Active BoNT/A and BoNT/A toxoid were obtained from Metabiologicals (Madison, Wis.) as a solution (0.25 mg/ml) in 0.01M phosphate buffer, pH 7.2 containing 0.15M NaCl (PBS) and 25% glycerol to prevent freezing and were stored at −20° C. The BoNT/A peptides (see Table 1) were synthesized by solid-phase peptide synthesis, purified and characterized as previously disclosed (Atassi et al., 1996; Atassi and Dolimbek, 2004).

Table 1 lists peptides of BoNT/A H chain. mPEG was coupled to each of these peptides at its N-terminal amino group for use in the present tolerization studies.

TABLE 1

| Peptide | | Residues of |
|---|---|---|
| Number | Amino Acid Sequence | SEQ ID NO: 1 |
| N8 | K Y T M F H Y L R A Q E F E H G K S R | 547-565 |
| N25 | N K F L N Q C S V S Y L M N S M I P Y | 785-803 |
| C15 | N N I M F K L D G C R D T H R Y I W I | 1051-1069 |
| C31 | S R T L G C S W E F I P V D D G W G E R P L | 1275-1296 |

Synthesis of the mPEG-Peptide Conjugates

Coupling of the peptides N8, N25, C15 and C31, to monomethoxypolyethylene glycol (mPEG) (molecular weight 5000, Aldrich) was carried out as described earlier (Atassi and Manshouri, 1991). Briefly, to 0.18 mMole peptide-resin, with N8, N25, C15 or C31 still attached to the solid support and with the N-terminal amino group of the peptide unprotected, 0.6 mMole mPEG succinate, 0.6 mMole 1-hydroxybenzotriazole monohydrate (Peptides International, Louisville, Ky.) and 0.6 mMole N,N′-diisopropylcarbodiimide (Fisher Scientific, Waltham, Mass.) in 10 ml mixture of equal volumes of dichloromethane (DCM) and dimethylformamide (DMF) were added and reacted for 96 h when negative ninhydrin reaction (Kaiser et al., 1970) was obtained. The peptide-mPEG conjugates were cleaved from the resin as described (Albericio et al., 1990) and the conjugate was separated from any uncoupled peptide by procedures described elsewhere (Atassi and Manshouri, 1991).

Tolerization and Immunization

The mice were allowed to rest for one week prior to use in the experiments. Tolerization of mice before immunization with BoNT/A and during the ongoing immune response was performed with the individual mPEG-peptide conjugates. Eleven, seven and three days (days −11, −7, −3) before immunization with BoNT/A toxoid, the mice (10 per group) were injected i.p. with 10 μg or with 30 μg of N8-mPEG in 20 μl PBS, N25-mPEG, C15-mPEG, or C31-mPEG. On day 0, the mice were immunized subcutaneously (s.c.) in the right footpad with 1 μg of BoNT/A toxoid in 10 μl suspension containing equal volumes of PBS and complete Freund's adjuvant (CFA). Control mice were not given any conjugate, but were immunized with BoNT/A toxoid. On day 21, the mice in all five groups were reimmunized (booster 1) with 1 μg of BoNT/A toxoid in 10 μl suspension containing equal volumes of PBS and incomplete Freund's adjuvant (IFA) in the left hind footpad, and ten days later a test bleed was taken (test bleed 1). A second booster was given on day 42 with the same amount of BoNT/A toxoid (1 μg dissolved in 10 μl mixture of equal volumes of PBS and IFA) on the right hind footpad, and ten days later (day 52) the animals were test bled (test bleed 2). The mice were rested for 69 days then on days 121, 125 and 129 they again received i.p. injections with the correlate mPEG-peptide in the same manner as on days −11, −7 and −3. The mice were given a third booster on day 132 with 1 μg BoNT/A (in 10 μl PBS/IFA) and bled ten days later (day 142; test bleed 3). Control group 5 received only BoNT/A toxoid.

Solid-Phase Radioimmunoassay

Each synthetic peptide (1 mg) was dissolved in 0.1 ml DMF and diluted with PBS up to 1 ml (1 mg/ml). From this stock solution a 50 μg/ml working solution in PBS was prepared and 50 μl of each peptide solution (2.5 μg peptide/50 μl) was introduced into three wells of a flexible 96-well flat bottom polyvinyl chloride plate (Becton Dickinson, New Jersey). Active BoNT/A solution in PBS (0.5 μg/50 μl) and a solution of negative control unrelated protein (BSA) were also plated into triplicate wells. The plates were kept at room temperature overnight. After washing the plates five times with PBS, they were blocked for 1 h at 37° C. with 100 μl of 1% bovine serum albumin in PBS (BSA/PBS). The plates were washed five times with PBS and an aliquot (50 μl) of antiserum from each of the mouse groups prediluted to 1:50 (vol/vol) with 0.1% BSA/PBS, was pipetted into the peptide, active BoNT/A or BSA coated wells and allowed to react for 3 h at 37° C. followed by standing overnight at 4° C. The wells were again washed five times with PBS and incubated (2 hr at 37° C.) with 50 μl of affinity-purified rabbit anti-mouse IgG (H+L)+IgM (Mu chain) (Accurate Chemical & Scientific Corporation, New York, N.Y.) Abs prediluted to 1:500 (vol/vol) with 0.1% BSA/PBS. The plates were then washed five times with PBS, and I¹²⁵-labeled protein A (2×10⁵ cpm in 50 μl 0.1% BSA/PBS, Sigma, Saint Louis, Mo.) was added to each well and allowed to react for 2 h at room temperature. Finally, the plates were washed thoroughly to remove any unbound radiolabel and dried. Individual wells were then cut out, transferred into clean tubes, and their radioactivity counted in a gamma-counter (1277 Gamma Master, LKB, Finland). Results from three replicate analyses provided the average binding of the antisera of ten mice in each group from which the percent inhibition in Ab titers relative to control (group 5) was determined.

Mouse Protection Assay

Antisera dilutions of a mixture of equal volumes from tolerized and control mice were determined by a mouse protection assay (MPA) for their ability to protect mice against challenge with a lethal dose of BoNT/A. Before testing, we first determined the survival of outbred (ICR) mice (ICR mice untolerized with conjugates and unimmunized with BoNT/A toxoid) against intravenous injection in the tail of different doses of active BoNT/A in a group of five mice. The LD₁₀₀ of the BoNT/A preparation at which no mouse survived was 10.0 pg/mouse. Then, different dilutions of the antisera from mPEG conjugate-tolerized or control mice (in 0.5 ml 0.5% BSA/saline) were mixed with 5×1.05×LD₁₀₀ of active BoNT/A (52.5 pg) in 0.5 ml 0.5% BSA/saline and incubated for 1 h at 37° C., and then placed on ice. Five mice were injected intravenously in the tail with 0.2 ml each of the mixture of each dilution. The MPA control mice received 10.5 pg active BoNT/A in 0.1 ml 0.5% BSA/saline. The mice were examined 3 times a day for 6 days. All 5 mice in the MPA control group that received active BoNT/A failed to survive. In the case that the sera dilutions afforded full protection, the mice survived the challenge. In the case that the Abs in the sera dilutions were diluted to lower titers, few or no mice survived. The percentage of mice that survived the toxin challenge relative to the total mice per group was plotted as a function of antisera dilutions and the 50% survival point for each group was determined from the plot. The percent Ab decrease in a tolerized groups was calculated from Ab titers relative to the Ab titer observed in the control.

Effect of Pretreatment with mPEG-Peptides on Antibody Levels.

The effects of administration of mPEG-peptide on the Ab response to the correlate region were determined at (a) 10 μg of a given mPEG peptide administered i.p. into 10 mice, or (b) 30 μg of a given mPEG-peptide administered i.p. into 10 mice. This was followed by immunization with inactivated BoNT/A (1 μg) on days 0 and 21. Test bleeds on day 31 were assayed with RIA for Ab levels against the four correlate peptide regions, and the blocking activity of the antisera at various dilutions was determined.

Effect of Tolerization with Low Doses of mPEG-Peptide.

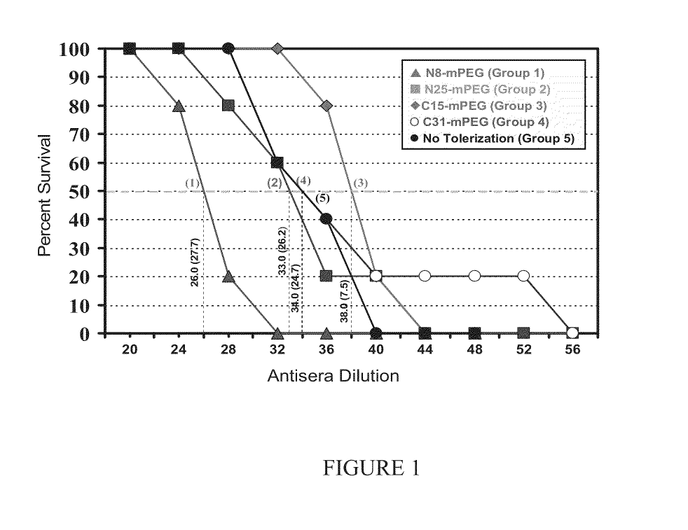

Pretreatment with mPEG-N8 caused approximately a 28% decrease in the Ab response to N8 relative to the response of control mice that had not been pretreated with any mPEG-peptide (FIG. 1, values in curly bracket). However it also caused a 24% decrease in the Ab response to N25 and a 22% decrease in the response to C31 (results not shown). Pretreatment with mPEG-N25 resulted in a 26% decrease in the Ab response to N25 and 39% and 19% decreases in the Ab responses to C15 and C31, respectively. Pretreatment with mPEG-C15 had little or no effect on the Ab responses to any of these regions, while mPEG-C31 caused the response to C31, N8 and C15 to be lower by 25%, 21% and 37%. These results indicated that with the exception of mPEG-C15, each of the other 3 mPEG-peptides was able to cause a substantial decrease in the Ab levels to the correlate region as well as other regions on the toxin.

We then determined the effect of the observed decrease in the levels on the protective activity of the antisera in vivo by MPA. The results are summarized in FIG. 1. Antisera obtained on day 31 from control mice that had been immunized with BoNT/A without treatment with any mPEG-peptide afforded complete protection against challenge with a lethal dose at 1:28 vol/vol after which protection declined, giving 50% protection at a dilution of 1:34 vol/vol. Antisera from mice that had been tolerized with mPEG-N8 were weaker and thus less protective exhibiting a 50% protection at a dilution of 1:26 vol/vol. Protection by the antisera from the other mouse groups that had been tolerized with mPEG-N25, mPEG-C15 or mPEG-C31 remained essentially the same as the control antisera.

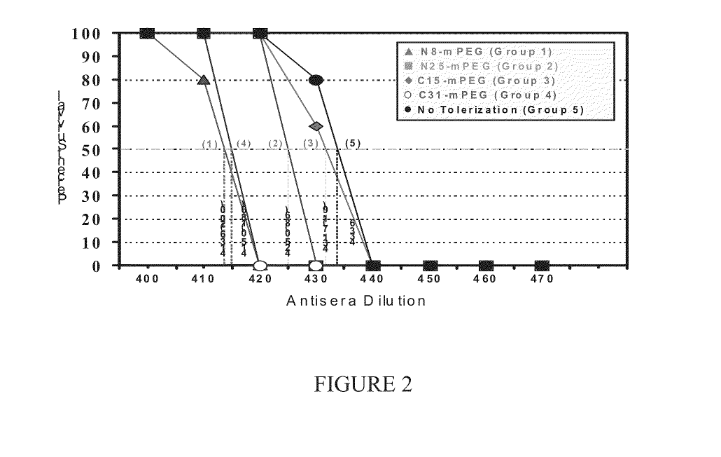

Antisera obtained on day 52 (test bleed 2) were also checked for their protective abilities in MPA (FIG. 2). Antisera from control untolerized mice gave 50% protection at a dilution of 1:434 vol/vol. Antisera from mPEG-C15 tolerized mice showed essentially similar protective activity with 50% survival at a dilution of 1:432 vol/vol. Mice tolerized with mPEG-N25 had somewhat weaker antisera that gave 50% protection at 1:425 vol/vol, while mice that were tolerized with mPEG-N8 or mPEG-C31 had even weaker antisera, which achieved 50% protection at dilution of 1:414 and 1:415 vol/vol, respectively.

Effect of Tolerization with Higher Doses of mPEG-Peptide.

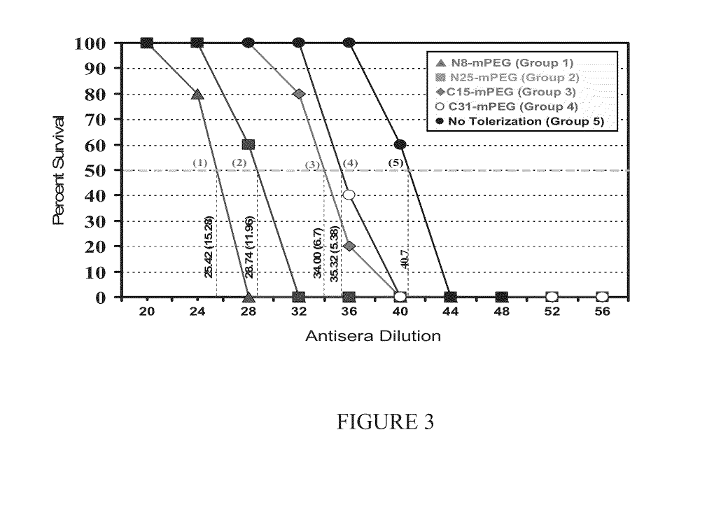

The effect of three injections of 30 μg per each of mPEG-peptide on the Ab levels was determined. Pretreatment with mPEG-N8 decreased the level of subsequent Ab responses to N8 (34%) (FIG. 3, values in curly bracket) as well as N25 (32%) and C31 (37%) relative to the response of control mice that had not been tolerized with any mPEG-peptide. Pretreatment with mPEG-N25 decreased the Ab responses to N25 (42%) and also to N8 (44%) and C15 (15%). mPEG-C15 lowered the Ab responses to C15 by 35% and also decreased the Ab responses to N8 (48%) and N25 (41%). Finally, pretreatment with mPEG-C31 lowered the Ab responses to C31 by 47% but also lowered the responses to N8 (47%) and N25 (31%). Thus it was found that a 30 μg dose of mPEG-peptide was quite effective in suppressing the Ab levels not only to the correlate peptide region but also to other regions on the toxin as well.

The effects on the protective activity of the antisera on day 31 post immunization (test bleed 1) are shown in FIG. 3. Antisera of un-tolerized controls achieved 50% protection at a dilution of 1:41 vol/vol. Antisera of mice that were tolerized with mPEG-C15 or mPEG-C31 were somewhat weaker, displaying 50% protection at dilutions of 1:34 and 1:35 vol/vol, respectively. However, mPEG-N25 or mPEG-N8 tolerized antisera were substantially weaker and gave 50% protection at dilutions of 1:29 and 1:25 vol/vol, respectively.

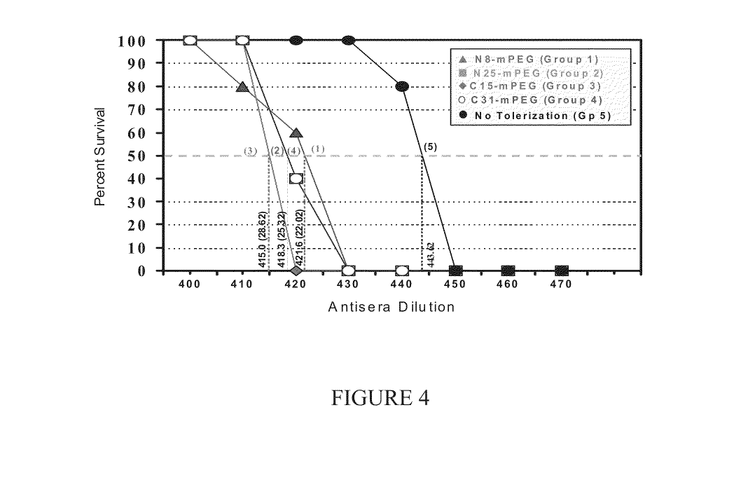

Antisera on day 52 (test bleed 2), presented in FIG. 4 from control un-tolerized mice showed 50% protection at a dilution of 1:444 vol/vol. In contrast, antisera from mPEG-C15, mPEG-N25, mPEG-C31 and mPEG-N8 tolerized mice were weaker and achieved 50% survival at a dilution of 1:415, 1:418, 1:418 and 1:422 vol/vol., respectively.

Long-Term Persistence of the Tolerance.

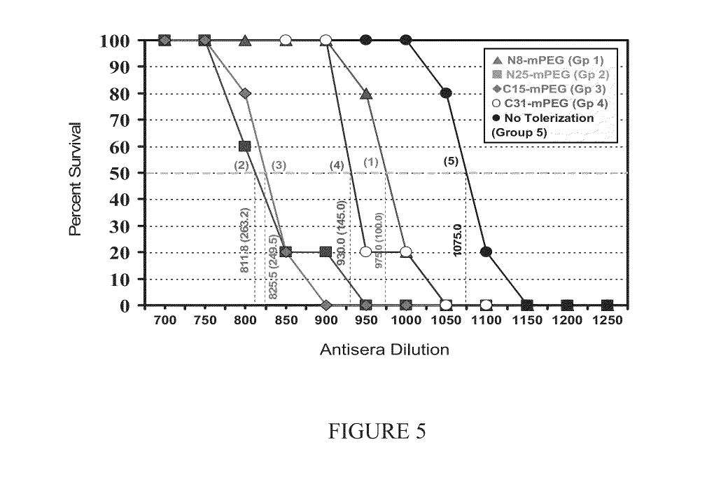

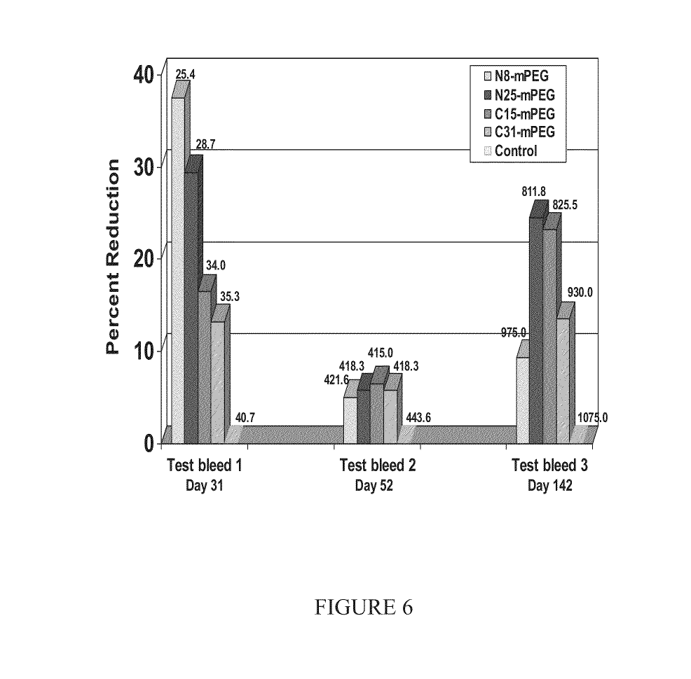

To determine whether suppression of the Ab levels observed after tolerization with a given mPEG-peptide could be maintained over a long duration, mice were tolerized with three 30 μg injections of a given mPEG-peptide as described in the preceding section. They were then allowed to rest for 69 days. On days 121, 125, and 129 mice were given i.p. injections of 30 μg each and then boosted on day 132 with 1 μg of BoNT/A. Antisera were obtained on day 142, assayed for Ab protective abilities in comparison to control untolerized mice determined by MPA. The results of long-term tolerance are shown in FIG. 5 and combined results for short-term and long-term tolerance experiments are summarized in FIG. 6. Control un-tolerized mice displayed 50% survival at a dilution of 1:1075 vol/vol. Tolerized mice had lower Ab titers and displayed 50% survival in MPA's at the following vol/vol dilutions of the antisera: mPEG-N25, 1:812; mPEG-C15, 1:826, mPEG-C31, 1:930 and mPEG-N8, 1:975.

The following lists the full citations for references cited above:

Albericio F., Kneib-Cordonier N., Biancalana S., Gera L., Masada R. I., Hudson D., Barany G. 1990. Preparation and application of the 5-(4-(9-fluorenylmethyloxycarbonyl)aminoethyl-3,5-dimethoxyphenoxy)-valeric acid (PAL) handle for the solid-phase synthesis of C-terminal peptide amides under mild conditions. J. Org. Chem., 55(12): 3730-3743.

Aoki K R. 2002. Immunologic and other properties of therapeutic botulinum toxin serotypes. In Scientific and Therapeutic Aspects of Botulinum Toxin, pp. 103-113. Edited by M. F. Brin, M. Hallett & J. Jankovic. Philadelphia: Lippincott Williams & Wilkins.

Aoki K R, Smith L A, Atassi M Z. 2010. Mode of action of botulinum neurotoxins. Current vaccination strategies and molecular immune recognition of the toxins. Crit Rev Immunol., (2010) 30(2):167-87.

Atassi M Z, Dolimbek B Z, Steward L E, Aoki K R. 2007. Molecular bases of protective immune responses against botulinum neurotoxin A—how antitoxin antibodies block its action. Crit Rev Immunol.; 27(4):319-41.

Atassi M Z, Dolimbek B Z, Hayakari M, Middlebrook J L, Whitney B, Oshima M. 1996. Mapping of the antibody-binding regions on botulinum neurotoxin H-chain domain 855-1296 with antitoxin antibodies from three host species. J Protein Chem. October; 15(7):691-700.

Atassi M Z, Dolimbek B Z. 2004. Mapping of the antibody-binding regions on the HN-domain (residues 449-859) of botulinum neurotoxin A with antitoxin antibodies from four host species. Full profile of the continuous antigenic regions of the H-chain of botulinum neurotoxin A. Protein J. January; 23(1):39-52.

Atassi M Z, Manshouri T. 1991. Synthesis of tolerogenic monomethoxypolyethylene glycol and polyvinyl alcohol conjugates of peptides. J Protein Chem. December; 10(6):623-627.

Atassi M Z, Oshima M (1999). Structure, activity, and immune (T and B cell) recognition of botulinum neurotoxins. Crit Rev Immunol. 19:219-60.

Atassi M Z, Ruan K H, Jinnai K, Oshima M, Ashizawa T. 1992. Epitope-specific suppression of antibody response in experimental autoimmune myasthenia gravis by a monomethoxypolyethylene glycol conjugate of a myasthenogenic synthetic peptide. Proc Natl Acad Sci USA. July 1; 89(13):5852-6.

Atassi M Z. 2004. Basic immunological aspects of botulinum toxin therapy. Mov. Disord. 19 (suppl 8), S68-84.

Atassi M Z, Dolimbek B Z, Steward L E, Aoki K R. 2010. Inhibition of botulinum neurotoxin A toxic action in vivo by synthetic synaptosome- and blocking antibody-binding regions. Manuscript prepared.

Comella C L, Jankovic J, Shannon K M, Tsui J, Swenson M, Leurgans S, Fan W; Dystonia Study Group. 2005. Comparison of botulinum toxin serotypes A and B for the treatment of cervical dystonia. Neurology 65, 1423-1429.

Dolimbek B Z, Aoki K R, Steward L E, Jankovic J, Atassi M Z. 2006. Mapping of the regions on the heavy chain of botulinum neurotoxin A (BoNT/A) recognized by antibodies of cervical dystonia patients with immunoresistance to BoNT/A. Mol Immunol. 2007 February; 44(5):1029-41. Epub May 2006.

Dolimbek B Z, Steward L E, Aoki K R, Atassi M Z. 2008. Immune recognition of botulinum neurotoxin B: antibody-binding regions on the heavy chain of the toxin. Mol Immunol. February; 45(4):910-24. Epub 2007 Sep. 25.

Dolimbek G S, Dolimbek B Z, Aoki K R, Atassi M Z. 2005. Mapping of the antibody and T cell recognition profiles of the HN domain (residues 449-859) of the heavy chain of botulinum neurotoxin A in two high-responder mouse strains. Immunol Invest. 34(2):119-42.

Dolimbek B Z, Steward L E, Aoki K R, Atassi M Z. 2010. Molecular immune recognition of botulinum neurotoxin A. The light chain regions recognized by anti-toxin antibodies and description of the complete antigenic structure of the toxin. Manuscript prepared.

Dressler D, Bigalke H, Benecke R. 2003. Botulinum toxin type B in antibody-induced botulinum toxin type A therapy failure. J. Neurol. 250, 967-969.

Dressler D, Bigalke H. 2004. Antibody-induced failure of botulinum toxin type B therapy in de novo patients. Eur. Neurol. 52, 132-135.

Göschel H, Wohlfarth K, Frevert J, Dengler R, Bigalke H. 1997. Botulinum A toxin therapy: neutralizing and nonneutralizing antibodies—therapeutic consequences. Exp. Neurol. 147, 96-102.

Greene P, Fahn S, Diamond B. 1994. Development of resistance to botulinum toxin type A in patients with torticollis. Mov. Disord. 9, 213-217.

Hamajima S, Atassi M Z. 1998. B-cell activation in vitro by helper T cells specific to a protein region that is recognized both by T cells and by antibodies. Immunol Invest. May; 27(3):121-34.

Jankovic J. 2002. Botulinum toxin: clinical implications of antigenicity and immunoresistance. In: Brin M F, Hallett M, Jankvoci J (Eds.), Scientific and therapeutic aspects of botulinum toxin. Lippincott Williams and Wilkins, Philadelphia, pp. 409-415.

Jankovic J. 2004. Treatment of cervical dystonia with botulinum toxin. Mov. Disord.; 19 (Suppl. 8):S109-115.

Kaiser E., Colescott R. L., Bossinger C. D., Cook P. I. 1970. Color test for detection of free terminal amino groups in the solid-phase synthesis of peptides. Anal. Biochem., April. 34(2): 595-598.

Maruta T, Dolimbek B Z, Aoki K R, Atassi M Z. 2006 Inhibition by human sera of botulinum neurotoxin-A binding to synaptosomes: A new assay for blocking and non-blocking antibodies. J. Neuroscience Methods. 151:90-96.

Maruta T, Dolimbek B Z, Aoki K R, Steward L E, Atassi M Z. 2004. Mapping of the synaptosome-binding regions on the heavy chain of botulinum neurotoxin A by synthetic overlapping peptides encompassing the entire chain. Protein J. 23:539-552.

Naumann M, Toyka K V, Mansouri Taleghani B, Ahmadpour J, Reiners K, Bigalke H.. Depletion of neutralising antibodies resensitises a secondary non-responder to botulinum A neurotoxin. J. Neurol. Neurosurg. Psychiatry (1998) 65, 924-927.

Oshima M, Atassi M Z. 2000. T cells of mice treated with mPEG-myasthenogenic peptide conjugate are involved in protection against EAMG by stimulating lower pathogenic antibody responses. Autoimmunity. 32(1):45-55.

Rosenberg J S, Atassi M Z. 1997. Intersite helper function of T cells specific for a protein epitope that is not recognized by antibodies. Immunol Invest. June; 26(4):473-89.

Rosenberg J S, Oshima M, Atassi M Z. B-cell activation in vitro by helper T cells specific to region alpha 146-162 of Torpedo californica nicotinic acetylcholine receptor. J Immunol. 1996 Oct 1; 157(7):3192-9

Sloop R R, Cole B A, Escutin R O. 1997. Human response to botulinum toxin injection: type B compared with type A. Neurology. 49, 189-194.

What is claimed is:

1. A method for reducing antibody response against botulism neurotoxin (BoNT) and BoNT variants comprising:
a) providing a suitable subject;
b) inoculating the subject with a monomethoxypolyethylene glycol-peptide conjugate;
wherein the monomethoxypolyethylene glycol-peptide conjugate comprises a BoNT-derived peptide; and,
wherein the BoNT-derived peptide is selected from the group consisting of: amino acids 547-565 of SEQ ID NO: 1; amino acids 785-803of SEQ ID NO: 1; amino acids 1051-1069 of SEQ ID NO: 1; and, amino acids 1275-1296 of SEQ ID NO: 1.

2. The method of claim 1, wherein the subject is a mammal.

3. The method of claim 1, wherein the subject is a human.
